(19) United States
(12) Patent Application Publication
(10) Pub. No.: US 2022/0205629 A1
(43) Pub. Date: Jun. 30, 2022

(54) LAMP FOR REMOVAL OF FINE DUST

(71) Applicant: PURUM AIR INC., Seoul (KR)

(72) Inventor: Chungho LEE, Anyang-si (KR)

(73) Assignee: PURUM AIR INC., Seoul (KR)

(21) Appl. No.: 17/601,394

(22) PCT Filed: Apr. 3, 2020
(86) PCT No.: PCT/KR2020/004583

§ 371 Date: Oct. 4, 2021

(30) Foreign Application Priority Data
Apr. 5, 2019 (KR) 10-2019-0040402

(51) Int. Cl. F21V 33/00; F21K 9/232; A61L 9/22

(52) U.S. Cl. CPC F21V 33/0088; F21Y 2115/10; A61L 9/22; F21K 9/232

(57) ABSTRACT

The present invention relates to a lamp for removal of fine dust. Disclosed according to an embodiment of the present invention is a lamp for removal of fine dust, which provides an indoor lighting function by using an LED as a light source and provides a function of adsorbing and removing fine dust by using an anion generator, wherein the lamp is configured to prevent the blackening effect caused when fine dust adsorbed and coagulated by anions emitted from the anion generator is suspended and adsorbed to a ceiling or wall.

### TECHNICAL FIELD

The present invention relates to a lamp for removal of fine dust, and more particularly, to a lamp for removal of fine dust, which provides an indoor lighting function by using an LED as a light source and provides a function of adsorbing and removing fine dust by using an anion generator, wherein the lamp is configured to prevent a blackening phenomenon caused when fine dust adsorbed and coagulated by anions emitted from an anion generator is suspended and adsorbed to a ceiling or a wall.

### BACKGROUND ART

Recently, air pollution caused by fine dust has occurred frequently, which is a great concern for human health.

In general, particles with a diameter of less than 10 micrometers are called fine dust. The fine dust mainly includes carbon, organic hydrocarbon, nitrate, sulfate, harmful metal components, and the like, which are combustion particles. They are so small that they may travel through a nose and a respiratory tract to reach the alveoli deep in the respiratory tract, and as they become smaller, they may pass directly through the alveoli for systemic circulation through the blood. Particles with a diameter of 2.5 micrometers or less are further classified as ultrafine dust.

An anion generator has been proposed as an apparatus for removing various harmful bacteria, dust, and fine dust present in indoor air. The anion generator may emit anions through a metal fiber exposed to the air, and may allow cationic particles such as dust or fine dust in the air to be adsorbed, coagulated, and settled with each other through the anions so that the cationic particles may be removed from the air.

As the related art proposed by the present inventor, Korean Patent Registration No. 10-0950713 (registered on Mar. 25, 2010), which has been filed prior to the present application and registered, relates to an LED lamp including an anion generator, in which an air flow is induced around a metal fiber by using a lens shade, and a photocatalytic coating layer is formed on a surface of the lens shade to minimize an amount of dust adsorbed while allowing heat generated during light emission of an LED to be easily dissipated to an outside, so that a lifespan of the LED may be extended.

However, while the related art provides an excellent dust adsorption and removal effect, the related art may cause a blackening phenomenon that makes a ceiling surface black as some of dust particles upon the adsorption and coagulation escape to an outside of the lens shade so as to be suspended in the air and adsorbed to the ceiling surface where the lamp is installed.

### DETAILED DESCRIPTION OF THE INVENTION

### Technical Problem

The present invention has been made to solve the above-described problems of the related art, and an object of the present invention is to provide a lamp for removal of fine dust, which provides an indoor lighting function by using an LED as a light source and provides a function of adsorbing and removing fine dust by using an anion generator, wherein the lamp is configured to prevent a blackening phenomenon caused when fine dust adsorbed and coagulated by anions emitted from an anion generator is suspended and adsorbed to a ceiling or a wall.

### Technical Solution

In order to achieve the object described above, according to one aspect of the present invention, a lamp for removal of fine dust includes: a body having a hollow shape, formed at an upper portion thereof with a closed surface on which a coupling socket terminal is installed, and having a bottom surface that is opened; an anion generator installed inside the body; an LED module installed inside the body, and installed such that an LED emits light through the bottom surface of the body; a lens unit installed on a lower portion of the body, and configured to transmit the light emitted from the LED downward; a metal fiber extending downward from the anion generator to penetrate centers of the LED module and a lens surface of the lens unit so as to be exposed to the lower portion of the body; and a lens shade coupled to a lower side of the body, and having a shape in which an upper circumference is narrower than a lower circumference, wherein a partition wall extending toward an inner space of the lens shade is provided along a circumference of the lens unit, and an inner upper space of the lens shade is divided into a partition wall inner space and a partition wall outer space by the partition wall.

Preferably, the partition wall may be formed integrally with the lens unit along a circumference of the lens surface of the lens unit.

Preferably, the lens unit may include: a ring-shaped coupling part inserted and coupled to a lower outer side of the body; the lens surface formed integrally with the ring-shaped coupling part on an inner side of the ring-shaped coupling part; and the partition wall formed integrally with the lens surface along the circumference of the lens surface.

Preferably, the partition wall may be formed on a ring-shaped partition wall member coupled to an outer side of the lens unit.

Preferably, the lens unit may include: a ring-shaped coupling part inserted and coupled to a lower outer side of the body; and the lens surface formed integrally with the ring-shaped coupling part on an inner side of the ring-shaped coupling part, and the ring-shaped partition wall member may include: a partition wall member coupling part inserted and coupled to an outer side of the ring-shaped coupling part of the lens unit; and the partition wall formed integrally with the partition wall member coupling part along a lower circumference of the partition wall member coupling part.

Preferably, the lens shade may include: a ring-shaped lens shade coupling part inserted and coupled to a lower outer side of the body; and a shade-shaped part expanding downward from a lower circumference of the ring-shaped lens shade coupling part.

Preferably, the present invention may further include an expanded circumference part expanding further outward along a lower circumference of the shade-shaped part.

### Advantageous Effects

As described above, according to the present invention, the indoor lighting function and the function of adsorbing and removing the fine dust by using the anion generator can be provided by a single lamp, and the blackening phenomenon caused when the fine dust adsorbed and coagulated by the anions emitted from the anion generator is suspended and adsorbed to the ceiling or the wall can be prevented.

### DESCRIPTION OF THE DRAWINGS

FIG. 1 is a perspective view showing a lamp for removal of fine dust according to one embodiment of the present invention.

FIG. 2 is a sectional view showing the lamp for the removal of the fine dust according to one embodiment of the present invention.

FIG. 3 is an exploded perspective view showing the lamp for the removal of the fine dust according to one embodiment of the present invention.

FIG. 4 is a bottom view showing the lamp for the removal of the fine dust according to one embodiment of the present invention.

FIG. 5 is a sectional view showing a lamp for removal of fine dust according to another embodiment of the present invention.

### BEST MODE

The present invention may be implemented in various other forms without departing from the technical idea or main features of the present invention. Therefore, embodiments of the present invention are merely for illustrative purposes in all respects, and should not be construed as limiting.

Terms such as “first” and “second” are used only to distinguish one element from another element. For example, a first element may be termed as a second element, and similarly, a second element may be termed as a first element, without departing from the scope of the present invention.

When one element is described as being “connected” or “accessed” to another element, it shall be construed as being connected or accessed to the other element directly, but also as possibly having another element in between.

As used herein, unless the context explicitly indicates otherwise, expressions in a singular form include a meaning of a plural form. In the present disclosure, a term such as “comprising”, “including”, or “having” is intended to designate the presence of elements or combinations thereof described herein, and shall not be construed to preclude any possibility of the presence or addition of other elements or characteristics.

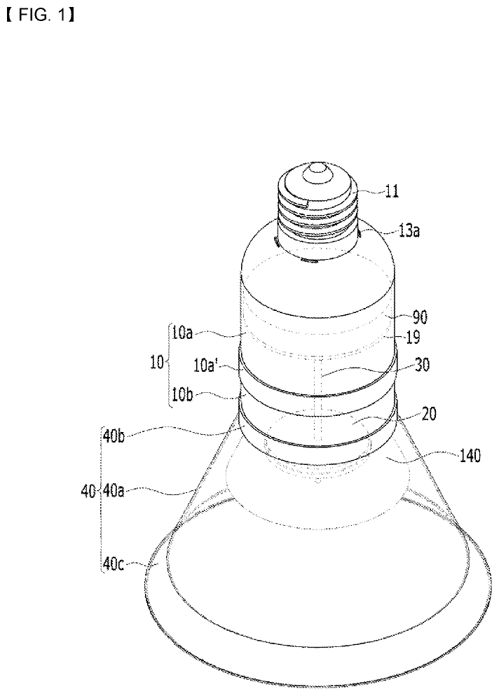

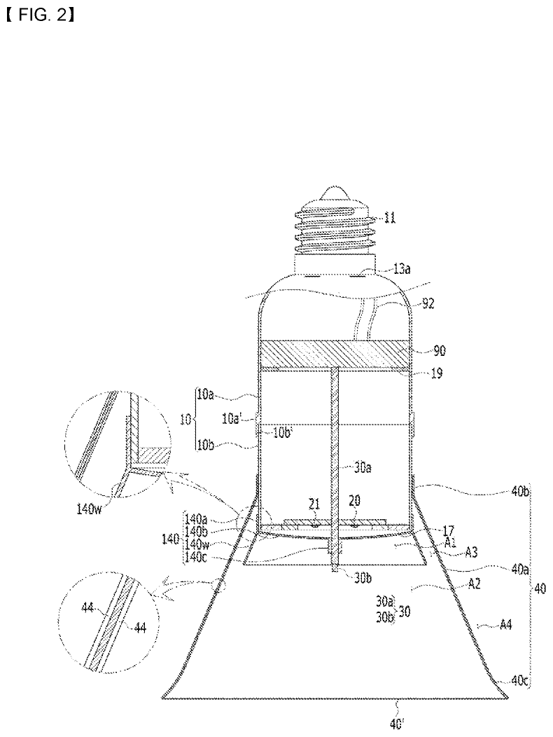

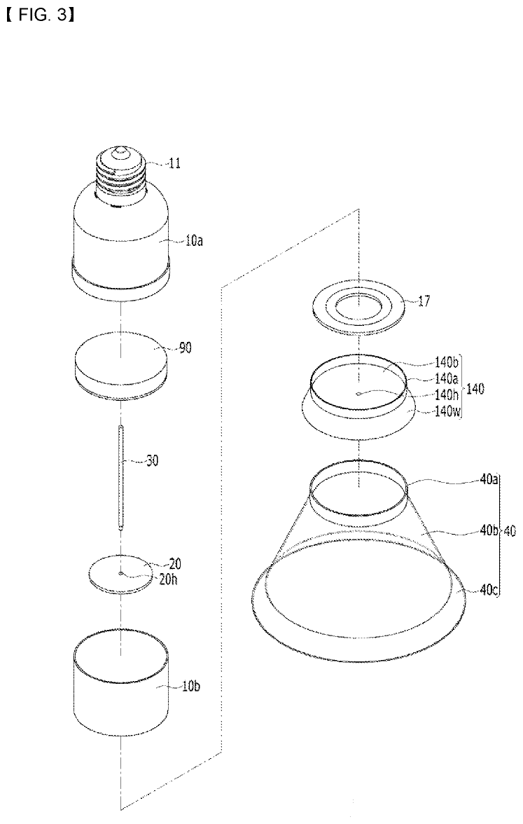

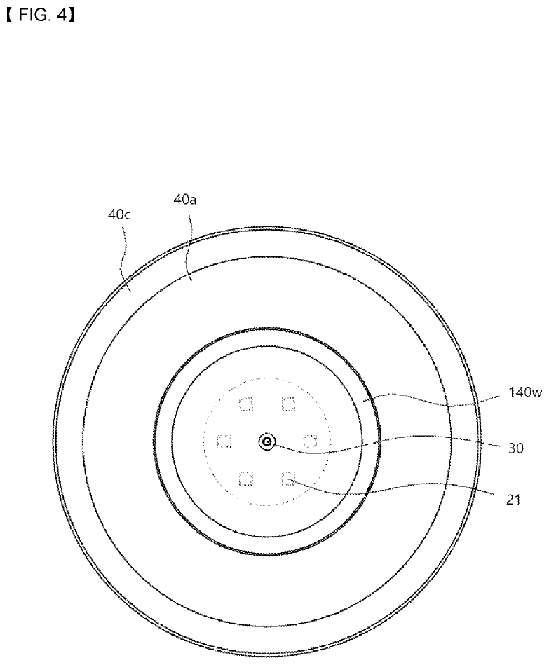

FIG. 1 is a perspective view showing a lamp for removal of fine dust according to one embodiment of the present invention, FIG. 2 is a sectional view showing the lamp for the removal of the fine dust according to one embodiment of the present invention, FIG. 3 is an exploded perspective view showing the lamp for the removal of the fine dust according to one embodiment of the present invention, and FIG. 4 is a bottom view showing the lamp for the removal of the fine dust according to one embodiment of the present invention.

According to the present embodiment, a body 10 of a lamp for removal of fine dust may be formed at an upper portion thereof with a closed surface (not denoted with a reference numeral) on which a coupling socket terminal 11 is installed, and may have a bottom surface (not denoted with a reference numeral) that is opened.

The body 10 may have a hollow shape formed of a synthetic resin material, and may be formed therein with an accommodation space so that an anion generator 90 may be mounted inside the body 10 and an LED module 20 may be mounted on a lower side of the anion generator 90, and an LED 21 of the LED module 20 may be exposed through the opened bottom surface. For example, the body 10 may have a cylindrical shape.

The socket terminal 11 may be installed on a top surface of the body 10 to supply a necessary power to the anion generator 90 and the LED module 20. The socket terminal 11 may be formed on an outer surface thereof with a spiral screw thread so as to be installed in an existing socket to which an incandescent light bulb is mounted. A plurality of heat dissipation holes 13a may be formed at a predetermined interval under a portion where the socket terminal 11 is formed along an outer circumferential surface of the body 10 having the cylindrical shape, so that air having a temperature that has been increased by the LED module 20 installed inside the body may be discharged to an outside.

The body 10 may be divided into an upper body 10a and a lower body 10b in consideration of convenience in assembly, which may be coupled integrally with each other by insertion coupling or screw coupling. Undescribed reference numerals 10a′ and 10b′ denote insertion coupling parts of the upper body 10a and the lower body 10b, respectively.

The body 10 may be provided therein with an installation member 19 for fixedly installing the anion generator 90 that will be described below, and another installation member 17 for fixedly installing the LED module 20. These installation members may have, for example, a ring shape so as to be fixed inside the body 10 by using various known coupling schemes including press-fitting, fusion bonding, or mechanical fastening.

The anion generator 90 may be installed inside the body 10. The anion generator 90 may include a substrate on which an anion circuit is formed.

The anion generator 90 may have various known configurations. For example, the anion generator 90 may generate anions through the following configuration to emit the anions through a bottom tip 30b of a metal fiber 30 that will be described below.

When a power is supplied from a DC power supply unit, an oscillation unit including a capacitor may oscillate to generate an output pulse having a predetermined frequency. A boosting unit including a resistor and a transistor may convert a DC voltage having a predetermined frequency that is output from the oscillation unit into a high voltage.

A high voltage unit in which a plurality of capacitors and diodes are connected in series and parallel may generate anions by boosting the output voltage of the boosting unit to the high voltage required to generate the anions while generating a negative voltage with a voltage value that allows an insulation resistance of air to be broken.

An anion emission unit may be driven by the high voltage output from the high voltage unit, and a high negative voltage may be generated in the metal fiber 30 connected to an electric discharge wire drawn to the outside, so that the anions may be generated and discharged.

The LED module 20 may be installed inside the body 10. For example, the LED module 20 may be configured such that a plurality of LEDs 21 are mounted on a bottom surface of a PCB substrate having a disc shape. The LED module 20 may be installed such that the LED 21 emits light through the bottom surface of the body 10.

A through-hole 20h may be formed vertically through a central portion of the LED module 20, so that through the through-hole 20h, the metal fiber 30 that will be described below may protrude from the anion generator, which is located on an upper portion of the LED module 20 within the body 10, to the outside on a lower portion of the body 10 through the LED module 20.

A lens unit 140 may be installed on the lower portion of the body 10. The lens unit 140 may be configured to transmit the light emitted from the LED 21 downward. For example, the lens unit 140 may be configured as a convex lens, and may be formed of a transmissive material having excellent thermal conductivity to facilitate heat dissipation.

The lens unit 140 may be formed at a center thereof with a through-hole 140h and a guide part 140c. The metal fiber 30 drawn out from the body 10 through the guide part 140c may protrude to a lower portion of the lens unit 140. Preferably, a length of the bottom tip of the protruding metal fiber 30 may be restricted so as to be present within the lens shade 40.

The guide part 140c may have a shape protruding from a bottom surface of the lens unit 140, so that a coated metal fiber 30 may be firmly supported by the guide part 140c without being bent.

The metal fiber 30 may be drawn out from the anion generator 90 to extend downward, and may penetrate centers of the LED module 20 and a lens surface 140b of the lens unit 140 so as to be exposed to the lower portion of the body 10.

For example, the metal fiber 30 may be coated with nano-silver, and then coated with a synthetic resin, in which an end of the metal fiber 30 may not be coated with the synthetic resin so as to expose the metal fiber 30 therein to the outside, so that the anions generated from the anion generator 90 may be discharged into the air. Preferably, the bottom tip of the metal fiber 30 may be detachably coupled so as to be replaced when fine dust adheres.

The lens shade 40 may be coupled to a lower side of the body 10, and may have a shape in which an upper circumference is narrower than a lower circumference, and a bottom surface 40′ of the lens shade 40 may be opened. For example, in consideration of an anion emission effect, the lens shade 40 may have a ratio such that a diameter of an upper portion is about 4 cm, a length is about 6 to 7 cm, and a diameter of a lower portion is about 9 to 10 cm.

In addition, the lens shade 40 may be formed on a surface thereof with a photocatalytic coating layer 44 formed of a titanium dioxide (TiO₂) material so that adsorbed and coagulated dust may not adhere to and contaminate the lens shade 40.

Preferably, the lens shade 40 may be formed by using a transparent polycarbonate material with a smooth surface and a high light transmittance to prevent glare caused by the light emitted from the LED module 20, and to increase light transmission efficiency in a lateral direction.

For example, the lens shade 40 may include: a ring-shaped lens shade coupling part 40b inserted and coupled to a lower outer side of the body 10; and a shade-shaped part 40a expanding downward from a lower circumference of the ring-shaped lens shade coupling part 40b. For example, the ring-shaped lens shade coupling part 40b may be coupled integrally to the lower outer side of the body 10 by the insertion coupling or the screw coupling.

Preferably, the lens shade 40 may further include an expanded circumference part 40c expanding further outward along a lower circumference of the shade-shaped part 40a. Since an inner space of the expanded circumference part 40c is a portion extending further outward from the LED module 20, the inner space of the expanded circumference part 40c may have a lower air temperature than a central space of the lens shade 40, and may provide a more advantageous environment for fine dust particles, which are suspended to move from an inner space A2 to an outside A4 of the lens shade 40, to be settled downward due to a low temperature.

A partition wall 140w extending toward the inner space A2 of the lens shade 40 may be provided along a circumference of the lens unit 140.

An inner upper space of the lens shade 40 may be divided into a partition wall inner space A1 and a partition wall outer space A3 by the partition wall 140w.

For example, the partition wall 140w may preferably extend to a height that is similar to a height of the bottom tip 30b of the metal fiber 30, or to a slightly lower position than the bottom tip 30b of the metal fiber 30. In addition, the partition wall 140w may be preferably formed in a direction parallel to the shade-shaped part 40a of the lens shade 40, or in a direction extending slightly inward therefrom.

For example, the partition wall 140w may be formed integrally with the lens unit 140 along a circumference of the lens surface 140b of the lens unit 140.

To this end, the lens unit 140 may include: a ring-shaped coupling part 140a inserted and coupled to a lower outer side of the body 10; the lens surface 140b formed integrally with the ring-shaped coupling part 140a on an inner side of the ring-shaped coupling part 140a; and the partition wall 140w formed integrally with the lens surface 140b along the circumference of the lens surface 140b. For example, the ring-shaped coupling part 140a may be coupled integrally with the lower outer side of the body 10 by the insertion coupling or the screw coupling.

When the partition wall 140w is formed in an integrated structure with the lens unit 140 as described above, the partition wall 140w may be formed together with the lens unit 140 upon manufacture of the lens unit 140 through an injection-molding scheme, so that it is advantageous in terms of convenience in manufacture.

According to the present embodiment, the lamp for the removal of the fine dust may provide a function of removing fine dust as follows.

When a power is applied while the coupling socket terminal 11 of the lamp for the removal of the fine dust is coupled to a socket (not shown) on a ceiling, the anion generator 90 may generate anions so that the anions may be emitted from the bottom tip of the metal fiber 30 to the inner space A2 of the lens shade 40 through the partition wall inner space A1.

Fine dust particles suspended in the air in a form of cation particles may be adsorbed and coagulated by the anions to form larger particles in the inner space A2 of the lens shade 40. When the fine dust particles are adsorbed to have a size that is as large as a size of normal dust particles, the fine dust particles may be settled on an indoor floor due to a weight of the particles, so that the fine dust particles suspended in the air may be settled and removed to the floor.

During the above process, fine dust located in the partition wall inner space A1 that is adjacent to the bottom tip of the metal fiber 30 may primarily make contact with the anions at a close distance, so that when compared with fine dust located in a space lower than the partition wall inner space A1, an adsorption and coagulation phenomenon may preferentially occur, and a particle size may be preferentially increased.

Meanwhile, since the partition wall inner space A1 is adjacent to the LED 21, heat generated from the LED 21 may be transmitted to the partition wall inner space A1, so that the partition wall inner space A1 may have a higher air temperature condition than the partition wall outer space A3.

Due to the above temperature condition, the fine dust particles that are adsorbed by the anions into larger particles in the partition wall inner space Al while being suspended without being settled on the indoor floor may ride over the partition wall 140w to move to the partition wall outer space A3.

However, since an air layer of the partition wall outer space A3 is not directly connected to an air layer of the partition wall inner space A1 due to the partition wall 140w, and the heat generated from the LED 21 is not directly transmitted to the partition wall outer space A3, the air layer of the partition wall outer space A3 may have a lower temperature than the air layer of the partition wall inner space A1.

Therefore, the fine dust particles riding over the partition wall 140w so as to be suspended to move from the partition wall inner space A1 to the partition wall outer space A3 may enter the air layer having a lower temperature condition, and remain in a space with an upper portion closed by the ring-shaped lens shade coupling part 40b of the lens shade 40, so that the fine dust particles may be settled downward without being suspended to an upper layer.

Since the fine dust particles moved to a central or a lower portion of the inner space A2 of the lens shade 40 continuously makes contact with the anions emitted from the bottom tip of the metal fiber 30, the adsorption and coagulation phenomenon may occur continuously, and the particle size may be increased. When the size of the fine dust particles becomes as large as the size of the normal dust particles, the fine dust particles may be settled on the indoor floor due to the weight of the particles, so that the fine dust particles suspended in the air may be settled and removed to the floor.

Through the above process, according to the present embodiment, the lamp for the removal of the fine dust may prevent a blackening phenomenon that makes a ceiling surface black as the fine dust is suspended to an outside of the lens shade 40 and adsorbed to the ceiling surface before the fine dust is adsorbed and coagulated to have a particle size that allows the fine dust to be settled.

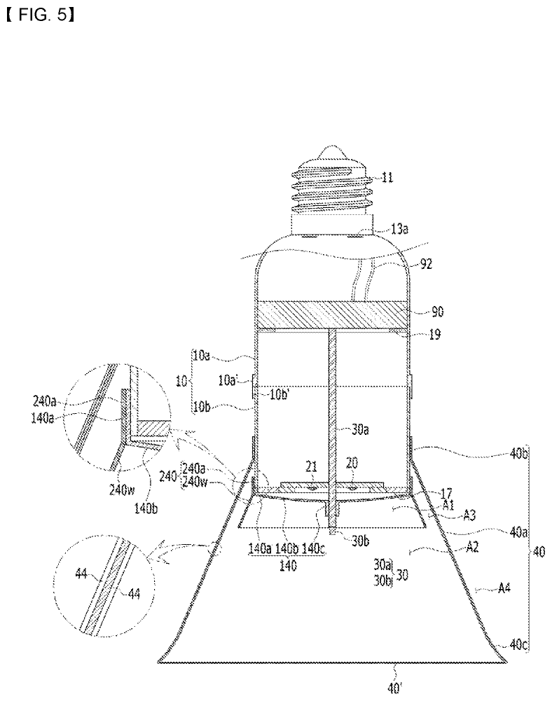

FIG. 5 is a sectional view showing a lamp for removal of fine dust according to another embodiment of the present invention.

While the partition wall 140w is formed integrally with the lens unit 140 in the above-described embodiment of FIGS. 1 to 4, a partition wall 240w may be formed on a ring-shaped partition wall member 240 coupled to an outer side of the lens unit 140 in the present embodiment.

To this end, according to the present embodiment, the lens unit 140 may include: a ring-shaped coupling part 140a inserted and coupled to a lower outer side of the body 10; and the lens surface 140b formed integrally with the ring-shaped coupling part 140a on an inner side of the ring-shaped coupling part 140a.

In addition, the ring-shaped partition wall member 240 may include: a partition wall member coupling part 240a inserted and coupled to an outer side of the ring-shaped coupling part 140a of the lens unit 140; and the partition wall 240w formed integrally with the partition wall member coupling part 240a along a lower circumference of the partition wall member coupling part 240a.

In general, the lens surface 140b of the lens unit 140 may be coated with a diffusion agent so that the light emitted from the LED 21 may be diffused to achieve an excellent lighting effect.

However, since the lamp for the removal of the fine dust according to the present embodiment is for a combined use including a function of a lighting device, when some of the diffusion agent is smeared on the partition wall 140w during a coating process of the diffusion agent, a diffusion agent coating layer may partially block light emission in the lateral direction so as to cause a partial loss of an illuminance.

In the case of the above-described embodiment of FIGS. 1 to 4, upon the coating of the diffusion agent, a separate cover treatment has to be performed so that the diffusion agent may not be smeared on the partition wall 140w that is formed integrally with the lens unit 140, or otherwise, a coating work has to be performed at a risk that some of the diffusion agent makes contact with the partition wall 140w.

According to the present embodiment, in consideration of the above configuration, the ring-shaped partition wall member 240 on which the partition wall 240w is formed and the lens unit 140 may be formed as separate members, so that possibility that the diffusion agent makes contact with the partition wall 240w may be fundamentally precluded when the lens surface 140b of the lens unit 140 is coated with the diffusion agent.

Although the present invention has been described with reference to the accompanying drawings based on exemplary embodiments, it will be apparent to those skilled in the art that many various and obvious modifications can be made from the above description without departing from the scope of the present invention. Therefore, the scope of the present invention should be interpreted by the appended claims described to encompass such many modifications.

1. A lamp for removal of fine dust, the lamp comprising:
a body having a hollow shape, formed at an upper portion thereof with a closed surface on which a coupling socket terminal is installed, and having a bottom surface that is opened;
an anion generator installed inside the body;
an LED module installed inside the body, and installed such that an LED emits light through the bottom surface of the body;
a lens unit installed on a lower portion of the body, and configured to transmit the light emitted from the LED downward;
a metal fiber extending downward from the anion generator to penetrate centers of the LED module and a lens surface of the lens unit so as to be exposed to the lower portion of the body; and
a lens shade coupled to a lower side of the body, and having a shape in which an upper circumference is narrower than a lower circumference,
wherein a partition wall extending toward an inner space of the lens shade is provided along a circumference of the lens unit, and
an inner upper space of the lens shade is divided into a partition wall inner space and a partition wall outer space by the partition wall.

2. The lamp of claim 1, wherein the partition wall is formed integrally with the lens unit along a circumference of the lens surface of the lens unit.

3. The lamp of claim 2, wherein the lens unit includes:
a ring-shaped coupling part inserted and coupled to a lower outer side of the body;
the lens surface formed integrally with the ring-shaped coupling part on an inner side of the ring-shaped coupling part; and
the partition wall formed integrally with the lens surface along the circumference of the lens surface.

4. The lamp of claim 1, wherein the partition wall is formed on a ring-shaped partition wall member coupled to an outer side of the lens unit.

5. The lamp of claim 4, wherein the lens unit includes:
a ring-shaped coupling part inserted and coupled to a lower outer side of the body; and
the lens surface formed integrally with the ring-shaped coupling part on an inner side of the ring-shaped coupling part, and the ring-shaped partition wall member includes:
a partition wall member coupling part inserted and coupled to an outer side of the ring-shaped coupling part of the lens unit; and
the partition wall formed integrally with the partition wall member coupling part along a lower circumference of the partition wall member coupling part.

6. The lamp of claim 1, wherein the lens shade includes:
a ring-shaped lens shade coupling part inserted and coupled to a lower outer side of the body; and
a shade-shaped part expanding downward from a lower circumference of the ring-shaped lens shade coupling part.

7. The lamp of claim 6, further comprising an expanded circumference part expanding further outward along a lower circumference of the shade-shaped part.
